(19) United States
(12) Patent Application Publication
(10) Pub. No.: US 2006/0009468 A1
(43) Pub. Date: Jan. 12, 2006

(54) Method for treatment of cancer

(76) Inventors: Peter A. Crooks, Nicholasville, KY (US); Robert Alonso, Philadelphia, PA (US)

Correspondence Address: ARLEN L. OLSEN;SCHMEISER, OLSEN & WATTS, 3 LEAR JET LANE, SUITE 201, LATHAM, NY, 12110, US

(21) Appl. No.: 11/176,852

(22) Filed: Jul. 7, 2005

Related U.S. Application Data
(60) Provisional application No. 60/586,315, filed on Jul. 7, 2004.

(51) Int. Cl. A61K 31/496

(52) U.S. Cl. 514/254.05

(57) ABSTRACT

A method for the treatment of cancer that includes in vivo delivering a sensitizer to a patient that has cancer; and exposing the patient to ionizing radiation, hyperthermia or an anticancer chemotherapeutic agent, or combinations of these agents. The sensitizer may be a structural analog of L-arginine, such as L-Canavanine or an ester prodrug of structural analogs of L-arginine, such as an ester of L-Canavanine. The chemotherapeutic agent may include DNA-interactive agents such as alkylating agents, e.g. cisplatin, cyclophosphamide, altretamine; DNA strand-breakage agents, such as bleomycin; intercalating topoisomerase II inhibitors, eg., dactinomycin and doxorubicin); nonintercalating topoisomerase II inhibitors such as, etoposide and teniposide; and the DNA minor groove binder plicamydin, for example.

The present patent application is a non-provisional application claiming priority from provisional application Ser. No. 60/586,315 (filed Jul. 7, 2004 and entitled “Method for Treatment of Cancer”).

### FIELD OF THE INVENTION

The present invention relates generally to methods for treating cancer patients, and more specifically to sensitizing cancer cells to cancer treatments.

### BACKGROUND

Radiation therapy uses high-energy rays directed at a tumor. This therapy damages the cancer cells and stops them from growing and dividing. It may be used before or after surgery to shrink the tumor alone or with chemotherapy for patients with inoperable tumors. Side effects include fatigue, skin becomes red, tender, itchy, nausea, vomiting, diarrhea, and/or digestion problems. The side effects usually subside when treatment ceases.

For patients with advanced cancer who cannot have their tumors removed surgically, the focus of treatment involves symptom prevention and control. This may involve the use of:

1. Surgery to relieve intestinal blockage or to perform nerve blocks for pain;

2. Radiation therapy to relieve painful disease sites; or

3. Chemotherapy to reduce the rate of tumor growth and to prolong survival

For some patients whose tumors cannot be removed surgically, chemotherapy and radiation therapy are sometimes given together to reduce the size of the tumor. There is a need for improved methods for treating patients who have cancer.

### SUMMARY OF THE INVENTION

A first aspect of the present invention provides a method for treatment of cancer, comprising: delivering in vivo a sensitizer to a cancer patient, and exposing the patient to a chemotherapeutic agent.

A second aspect of the present invention provides a pharmaceutical composition comprising a sensitizer and a pharmaceutically acceptable carrier.

A third aspect of the present invention provides a method, comprising: delivering in vivo a dose of a sensitizer to a patient that has cancer, wherein the sensitizer is represented by at least one of the following structures (a-i) of Formula 4 in its S—, R—, or racemic form: 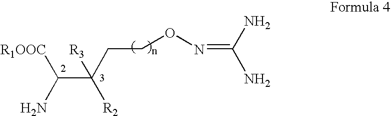
wherein
(a) R₁ and R₂═H; R₃═NH₂; n=0-3
(b) R₁ and R₂═H; R₃═NH₂; n=0-3
(c) R₁ and R₃═H; R₂═NH₂; n=0-3
(d) R₁=methyl; R₂═H; and R₃═NH₂; n=0-3
(e) R₁=ethyl; R₂═H; and R₃═NH₂; n=0-3
(f) R₁=isopropyl; R₂═H; and R₃═NH₂; n=0-3
(g) R₁=n-propyl; R₂═H; and R₃═NH₂; n=0-3
(h) R₁=n-butyl; R₂═H; and R₃═NH₂; n=0-3
(i) R₁=n-octyl; R₂═H; and R₃═NH₂; n=0-3 and exposing the patient to a chemotherapeutic agent.

A fourth aspect of the present invention is a method, comprising: delivering in vivo a dose of a sensitizer to a patient that has cancer, wherein the sensitizer is represented by at least one of the following structures (a-i) of Formula 5: 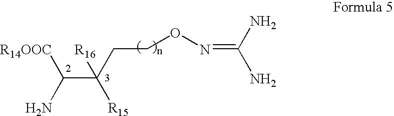
wherein
(a) R₁₄ and R₁₅═H; R₁₆═NH₂; n=0-3; 2R, 3S
(b) R₁₄ and R₁₅═H; R₁₆═NH₂; n=0-3; 2R, 3S
(c) R₁₄ and R₁₆═H; R₁₅═NH₂; n=0-3; 2R, 3R
(d) R₁₄ and R₁₅═H; R₁₆═NH₂; n=0-3; 2R, 3S
(e) R₁₄=ethyl; R₁₅═H; and R₁₆═NH₂; n=0; 2R, 3S
(f) R₁₄=isopropyl; R₁₅═H; and R₁₆═NH₂; n=0-3; 2R, 3S
(g) R₁₄=n-propyl; R₁₅═H; and R₁₆═NH₂; n=0-3; 2R, 3S
(h) R₁₄=n-butyl; R₁₅═H; and R₁₆═NH₂; n=0-3; 2R, 3S
(i) R₁₄=n-octyl; R₁₅═H; and R₁₆═NH₂; n=0-3; 2R, 3S and

exposing the patient to a chemotherapeutic agent.

A fifth aspect of the present invention is a method, comprising: delivering in vivo a dose of a sensitizer to a patient that has cancer, wherein the sensitizer is represented by at least one structure (a-h) of Formula 6, 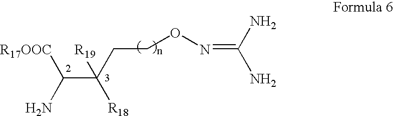
wherein
(a) R₁ and R₃═H; R₂═NH₂; n=0-3; 2R, 3R
(b) R₁ and R₃═H; R₂═NH₂; n=0-3; 2R, 3R
(c) R₁=methyl; R₃═H; and R₂═NH₂; n=0-3; 2R, 3R
(d) R₁=ethyl; R₃═H; and R₂═NH₂; n=0-3; 2R, 3R
(e) R₁=isopropyl; R₃═H; and R₂═NH₂; n=0-3; 2R, 3R
(f) R₁=n-propyl; R₃═H; and R₂═NH₂; n=0-3; 2R, 3R
(g) R₁=n-butyl; R₃═H; and R₂═NH₂; n=0-3; 2R, 3R
(h) R₁=n-octyl; R₃═H; and R₂═NH₂; n=0-3; 2R, 3R; and

exposing the patient to a chemotherapeutic agent.

A sixth aspect of the present invention is a method, comprising: delivering in vivo a dose of a sensitizer to a patient that has cancer, wherein the sensitizer is represented by by at least one structure (a-h) of Formula 7: 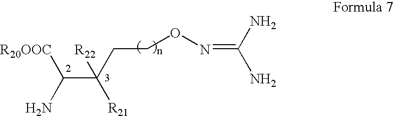
wherein
(a) R₁ and R₃═H; R₂═NH₂; n=0-3; 2S, 3R
(b) R₁ and R₃═H; R₂═NH₂; n=0-3; 2S, 3R
(c) R₁=methyl; R₃═H; and R₂═NH₂; n=0-3; 2S, 3R
(d) R₁=ethyl; R₃═H; and R₂═NH₂; n=0-73; 2S, 3R
(e) R₁=isopropyl; R₃═H; and R₂═NH₂; n=0-3; 2S, 3R
(f) R₁=n-propyl; R₃═H; and R₂═NH₂; n=0-3; 2S, 3R
(g) R₁=n-butyl; R₃═H; and R₂═NH₂; n=0-3; 2S, 3R
(h) R₁=n-octyl; R₃═H; and R₂═NH₂; n=0-3; 2S, 3R; and
   exposing the patient to a chemotherapeutic agent.

### BRIEF DESCRIPTION OF THE DRAWINGS

FIG. 1 depicts a method for the treatment of cancer, in accordance with embodiments of the present invention; and

FIG. 2 depicts an apparatus for delivering a sensitizer to a cancer, in accordance with embodiments of the present invention.

### DETAILED DESCRIPTION OF THE INVENTION

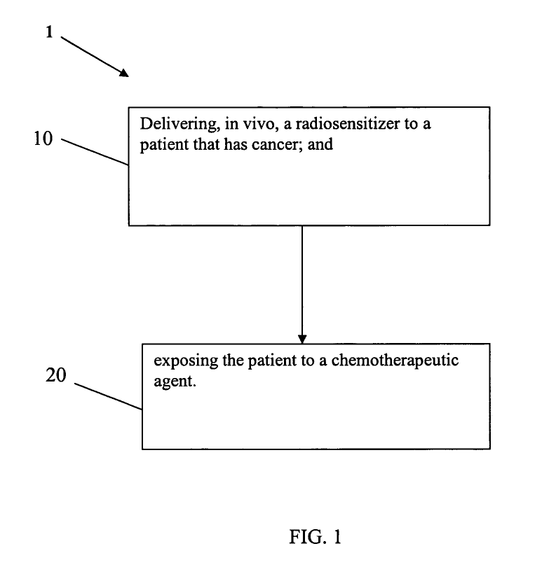

FIG. 1 depicts a method 1 for treatment of cancer, such as, for example, pancreatic cancer, said method 1 includes a step 10, delivering in vivo a dose of a sensitizer to a patient that has cancer; and a step 20, exposing the patient to a chemotherapeutic agent, or an ionizing radiation, or a heat source, or a combination of any of these three exposures. Hereinafter, “delivering in vivo a dose of a sensitizer to a patient” is defined as bringing or transporting or distributing to the proper place within the patient's body, such as to a cancer infected organ system, organ or tissue within the patient's body to cure the cancer or conduct metabolic studies in the patient's body. Hereinafter organ systems include the endocrine system, which includes the following organs: hypothalamus, pituitary, thyroid, pancreas and adrenal glands; and the hepatic system, which includes the following organs: liver, pancreas and gall bladder. The “patient” is defined as any animal such as any mammal, such as a person or human being who is the recipient of the sensitizer. Alternatively, delivering in vivo may mean conducting metabolic studies including delivering the sensitizer within the body of the animal.

The sensitizer may be L-arginine, and/or a structural analog of L-arginine such as a L-Canavanine, and/or a dihydrohalide salt or admixtures of the acid with a salt-forming material. The dihydrohalide salts may be dihydrofluoride, chloride, bromide or iodide, and combinations thereof. Alternatively, the sensitizer may be a carboxylic acid salt of L-arginine, and/or a structural analog of L-arginine such as a L-Canavanine, wherein the cation may be any metal such as, but not restricted to, sodium, potassium or calcium, and combinations thereof.

The sensitizer may be represented by Formula 1 in its S—, R—, or racemic form, as follows: 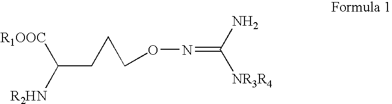
wherein R₁, R₃ and R₄ may independently at each occurrence be a hydrogen atom or a hydrocarbyl group, said hydrocarbyl group with a primary, a secondary or a tertiary carbon attachment point, selected from the group consisting of an alkyl group, an alkenyl group, an alkynl group, an aralkyl group, an alkaryl group and an aryl group.

The alkyl, alkenyl, alkynl, aralkyl, alkaryl or aryl groups may have from 1-20 carbon atoms.

The alkyl groups of the aralkyl, or alkaryl groups may be linear, branched or cyclic and the aryl groups may be at least one C₃-C₈ carbon ring.

Each R₂ and R₃ independently at each occurrence may be a hydrogen atom or a carbonyl. The carbonyl may include tert-butyloxycarbonyl (BOC-) and benzoyl (Bz-).

Examples of sensitizers include compounds represented by Formula 1, wherein R₁═H, CH₃or CH₃CH₂, R₂═H, tert-butyloxycarbonyl (BOC-), or benzoyl (Bz-), R₃═H, CH₃, tert-butyloxycarbonyl (BOC-), or benzoyl (Bz-) and R₄═H, CH₃, or CH₃CH₂.

Alternatively, the sensitizer may be selected from the group of sensitizers consisting of D-2-Amino-3-(aminooxy)propionic acid dihydrochloride; D-2-Amino-3-(guanidinooxy)propionic acid; L-2-Amino-4- [assym-N^{G}, N^{G}-dimethyl (guanidinooxy)] butanoic acid; and mixtures thereof.

Alternatively, the sensitizer may be derived from a prodrug selected from the group of prodrugs consisting of L-Canavanine esters, methyl L-2-amino-4-guanidinooxybutanoate, ethyl L-2-amino-4-guanidinooxybutanoate, isopropyl L-2-amino-4-guanidinooxybutanoate, n-propyl L-2-amino-4-guanidinooxybutanoate, n-butyl L-2-amino-4-guanidinooxybutanoate, n-octyl-4-guanidinooxybutanoate, and mixtures thereof. Said L-Canavanine Esters may include methyl, ethyl, isopropyl and n-propyl esters of L-Canavanine.

The sensitizer may be administered in vivo or in vitro as the dihydrochloride salt, in the salt form, in the form of the mono- or dihydrochloride salt, and as the prodrug of L-Canavanine selected from the group consisting of D-2-amino-3-(aminooxy)propionic acid dihydrochloride. The active agent may be an analog of L-arginine, such as L-Canavanine, and the method of treatment of cancer includes use of the active agent in the form of any suitable salt including any mono or dihydrohalide salt where the halide is fluoride, chloride, bromide, or iodide or any alkali metal salt. Alternatively, the sensitizer may be derived from a prodrug such as an ester of L-arginine, and/or a structural analog of L-arginine such as a L-Canavanine. A dose of the sensitizer or prodrug of the sensitizer for a mammal, such as a human being may be from about 25 to about 50 mg per kilogram body weight per day. Alternatively, a dose of the sensitizer or prodrug of the sensitizer for a mammal, such as a human being may be from about 0.1 to about 25 mg per kilogram body weight per day.

L-Canavanine, the naturally occurring non-protein,δ-oxa analog of L-arginine, may be found in a variety of higher plants. L-Canavanine's usefulness as a sensitizer is enhanced by its apparent cytoselective toxicity towards transformed cells. In particular, L-Canavanine has demonstrated the capacity to inhibit the growth of pancreatic cancers both in vitro and in vivo. L-Canavanine may have particular utility as a therapy for pancreatic cancer since it may be selectively taken up by the pancreas.

L-Canavanine may be incorporated in place of L-arginine into newly synthesized proteins in a wide variety of organisms, resulting in the formation of non-functional proteins. These non-functional proteins may be variously manifested as structural and functional defects, including morphological and developmental aberrations, altered protein conformation and structure, and impaired enzymatic activity, as well as decreased cellular tolerance to heat, radiation, and other stressors.

Referring to FIG. 1, in the steps 10 and 20 of the method 1, it has been the experience of the inventors that delivering in vivo a sensitizer to a patient that has cancer, such as, for example, pancreatic cancer, as in the step 10; and exposing the patient to a chemotherapeutic agent, as in the step 20 may be an effective adjunctive or adjunct therapy for treatment of patients with cancer. “Adjunctive” or “adjunct” therapies are used in conjunction with others. Most cancer patients are found to be deficient in selenium, so many doctors add this element to their protocol. Therefore, it would be considered an “adjunctive therapy.” Other adjunctive therapies would include: detoxification, specific vitamins and supplements such as Vitamin C and Co-Q 10, water therapy, and nutrition balancing.

It has been the experience of the inventors using in-vitro experiments, that sensitizers of the present invention increase the percentage of cells in the G₂/M phase of the cell cycle that are sensitive to chemotherapeutic agents, when compared to untreated cells. Hereinafter, in-vitro experiments are experiments in which the sensitizer is delivered to cancerous cells or normal cells of a mammal, such as a human for the purpose of determining the effect of the sensitizer on the cells, wherein the cells are removed from the body of the mammal. In-vitro experiments involve removing cells from the body of the mammal, such as a human being, and exposing them to the sensitizer for cancer prevention or metabolic studies outside the body of the mammal, such as a human being in contrast to in-vivo treatments or studies in which the sensitizer is introduced into or within the body of the mammal, such as the human being to treat or conduct metabolic studies on cancerous cells within the body. For example, Crooks et al. concluded from an in-vitro study that after 72 hrs of exposure to L-canavanine, the percentage of cells in the radiosensitive G₂/M phase of the cell cycle increased 6-fold in PANC-1 cells and 4-fold in MIA PaCa-2 cells, when compared to untreated cells. See Peter A. Crooks et al., “L-Canavanine as a Radiosensitization Agent for Human Pancreatic Cancer Cells,” Molecular and Cellular Biochemistry 244: 37-43, 2003, herein incorporated by reference. It has also been found that the in-vitro capacity of L-canavanine to redistribute cells into the G₂/M phase of the cell cycle was both concentration—and time-dependent. See Id.

Chemotherapeutic agents of the present invention to which the patient may be exposed may be any appropriate chemotherapeutic agent such as, for example, exposing the cancer patient to a chemotherapeutic agent or radiation. Chemotherapeutic agents are generally grouped as DNA-interactive agents, antimetabolites, tubulin-interactive agents, hormonal agents, other agents such as asparaginase or hydroxyurea, and agents as set forth in Table 1, herein. Each of the groups of chemotherapeutic agents can be further divided by type of activity or compound. Chemotherapeutic agents used in combination with a compound of the present invention, or salts thereof of the present invention may be selected from any of these groups but are not limited thereto. DNA-interactive agents include alkylating agents, e.g. cisplatin, cyclophosphamide, altretamine; DNA strand-breakage agents, such as bleomycin; intercalating topoisomerase II inhibitors, eg., dactinomycin and doxorubicin); nonintercalating topoisomerase II inhibitors such as, etoposide and teniposide; and the DNA minor groove binder plicamydin, for example.

The alkylating agents form covalent chemical adducts with cellular DNA, RNA, or protein molecules, or with smaller amino acids, glutathione, or similar chemicals. Generally, alkylating agents react with a nucleophilic atom in a cellular constituent, such as an amino, carboxyl, phosphate, or sulfhydryl group in nucleic acids, proteins, amino acids, or in glutathione. The mechanism and the role of these alkylating agents in cancer therapy is not well understood.

Typical alkylating agents include, but are not limited to, nitrogen mustards, such as chlorambucil, cyclophosphamide, isofamide, mechlorethamine, melphalan, uracil mustard; aziridine such as thiotepa; methanesulphonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine. The chemotherapeutic agent may be selected from the group consisting of: cisplatin, doxirubicin, danurubicin, tamoxiphen, taxol, endoxan, Xeloda (capecitabin), Busulfex (busulfan), doramycin, and methotrexate.

DNA strand breaking agents include bleomycin, for example.

DNA topoisomerase II inhibitors include the following intercalators, such as amsacrine, dactinomycin, daunorubicin, doxorubicin (adriamycin), idarubicin, and mitoxantrone; nonintercalators, such as etoposide and teniposide, for example.

A DNA minor groove binder is plicamycin, for example.

Antimetabolites interfere with the production of nucleic acids by one of two major mechanisms. Certain drugs inhibit production of deoxyribonucleoside triphosphates that are the immediate precursors for DNA synthesis, thus inhibiting DNA replication. Certain of the compounds are analogues of purines or pyrimidines and are incorporated in anabolic nucleotide pathways. These analogues are then substituted into DNA or RNA instead of their normal counterparts.

Antimetabolites useful herein include, but are not limited to, folate antagonists such as methotrexate and trimetrexate; pyrimidine antagonists, such as fluorouracil, fluorodeoxyuridine, CB3717, azacitidine, cytarabine, and floxuridine; purine antagonists include mercaptopurine, 6-thioguanine, fludarabine, pentostatin; and ribonucleotide reductase inhibitors include hydroxyurea.

Tubulin interactive agents act by binding to specific sites on tubulin, a protein that polymerizes to form cellular microtubules. Microtubules are critical cell structure units. When the interactive agents bind the protein, the cell can not form microtubules. Tubulin interactive agents include vincristine and vinblastine, both alkaloids and paclitaxel (Taxol), for example.

Hormonal agents are also useful in the treatment of cancers and tumors. They are used in hormonally susceptible tumors and are usually derived from natural sources. Hormonal agents include, but are not limited to, estrogens, conjugated estrogens and ethinyl estradiol and diethylstilbesterol, chlortrianisen and idenestrol; progestins such as hydroxyprogesterone caproate, medroxyprogesterone, and megestrol; and androgens such as testosterone, testosterone propionate; fluoxymesterone, and methyltestosterone.

Adrenal corticosteroids are derived from natural adrenal cortisol or hydrocortisone. They are used because of their anti-inflammatory benefits as well as the ability of some to inhibit mitotic divisions and to halt DNA synthesis. These compounds include, but are not limited to, prednisone, dexamethasone, methylprednisolone, and prednisolone.

Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes.

Antihormonal antigens include, for example, antiestrogenic agents such as tamoxifen, antiandrogen agents such as flutamide; and antiadrenal agents such as mitotane and aminoglutethimide.

Further agents include the following: hydroxyurea appears to act primarily through inhibition of the enzyme ribonucleotide reductase, and asparaginase is an enzyme which converts asparagine to nonfunctional aspartic acid and thus blocks protein synthesis in the tumor.

Taxol (paclitaxel) may be a chemotherapeutic agent.

Ethyol (amifostine), available from Alza Pharmaceuticals, U.S. Bioscience has been approved by the U.S. Food and Drug Administration (FDA) to reduce the renal toxicity associated with repeated administration of chemotherapy in subjects with advanced ovarian cancer. Currently, there are only limited data on the effects of Ethyol on the efficacy of chemotherapy in other settings. Ethyol should not be administered to patients receiving chemotherapy for malignancies that are commonly curable, except in the context of a clinical study. This medication may be used to reduce the risk of kidney problems caused by the use of cisplatin or to reduce dry mouth caused by radiation treatment. Alternatively, this drug may also be used for prevention of lung damage caused by the use of paclitaxel.

A non-limiting, and not meant to be inclusive listing of currently available chemotherapeutic agents, according to class, and including diseases for which the agents are indicated, is provided as Table 1, herein. See James, C. Quada, Jr., U.S. Pat. No. 6,720,349.

TABLE 1
Neoplastic Diseases' for which Exemplary Chemotherapeutic agents are Indicated

| Class | Type of Agent | Name | Disease |
|---|---|---|---|
| Alkylating Agents | Nitrogen Mustards | Mechlorethamine | Hodgkin's disease, |
| | | (HN₂) | non-Hodgkin's lymphomas |
| | | Cyclophosphamide Ifosfamide | Acute and chronic lymphocytic |
| | | | leukemias, Hodgkin's disease, |
| | | | non-Hodgkin's lymphomas, multiple |
| | | | myeloma, neuroblastoma, breast, |
| | | | ovary, lung, Wilms' tumor, |
| | | | cervix, testis, soft-tissue |
| | | | sarcomas |
| | | Melphalan | Multiple myeloma, breast, ovary |
| | | Chlorambucil | Chronic lymphocytic leukemia, |
| | | | primary marco globulinemia, |
| | | | Hodgkin's disease, |
| | | | non-Hodgkin's lymphomas |
| | | Estramustine | Prostate |
| | Ethylenimines and | Hexamethylmelamine | Ovary |
| | Methylmelamines | Thiotepa | Bladder, breast, ovary |
| | Alkyl Sulfonates | Busulfan | Chronic granulocytic leukemia |
| | Nitrosoureas | Carmustine | Hodgkin's disease, non-Hodgkin's |
| | | | lymphomas, primary brain tumors, |
| | | | multiple myeloma, malignant |
| | | | melanoma |
| | | Lomustine | Hodgkin's disease, non-Hodgkin's |
| | | | lymphomas, primary brain |
| | | | tumors, small-cell lung |
| | | Semustine | Primary brain tumors, stomach, |
| | | | colon |
| | | Streptozocin | Malignant pancreatic insulinoma, |
| | | | malignant carcinoid |
| | Triazenes | Dacarbazine | Malignant melanoma, Hodgkin's |
| | | | disease, soft-tissue sarcomas |
| | | Procarbazine | |
| | | Aziridine | |
| Antimetabolites | Folic Acid | Methotrexate | Acute lymphocytic leukemia, |
| | Analogs | Trimetrexate | choriocarcinoma, mycosis |
| | | | fungoides, breast, head and |
| | | | neck, lung, osteogenic sarcoma |
| | Pyrimidine Analogs | Fluorouracil | Breast, colon, stomach, pancreas, |
| | | Floxuridine | ovary, head and neck, urinay |
| | | | bladder, premalignant skin |
| | | | lesions (topical) |
| | | Cytarabine Azacitidine | Acute granulocytic and acute |
| | | | lymphocytic leukemias |
| | Purine Analogs and | Mercaptopurine | Acute lymphocytic, acute |
| | Related Inhibitors | | granulocytic, and chronic |
| | | | granulocytic leukemias |
| | | Thioguanine | Acute granulocytic, acute |
| | | | lymphocytic, and chronic |
| | | | granulocytic leukemias |
| | | Pentostatin | Hairy cell leukemia, mycosis |
| | | | fungoides, chronic lymphocytic |
| | | | leukemia |
| | | Fludarabine | Chronic lymphocytic leukemia, |
| | | | Hodgkin's and non-Hodgkin's |
| | | | lymphomas, mycosis fungoides |
| Natural Products | Vinca Alkaloids | Vinblastine (VLB) | Hodgkin's disease, non-Hodgkin's |
| | | | lymphomas, breast, testis |
| | | Vincristine | Acute lymphocytic leukemia, |
| | | | neuroblastoma, Wilms' tumor, |
| | | | rhabdomyosarcoma, Hodgkin's |
| | | | disease, non-Hodgkin's |
| | | | lymphomas, small-cell lung |
| | | Vindesine | Vinca-resistant acute lymphocytic |
| | | | leukemia, chronic myelocytic |
| | | | leukemia, melanoma, lymphomas, |
| | | | breast |
| | Epipodophyllotoxins | Etoposide | Testis, small-cell lung and |
| | | Teniposide | other lung, breast, Hodgkin's |
| | | | disease, non-Hodgkin's |
| | | | lymphomas, acute, granulocytic |
| | | | leukemia, Kaposi's sarcoma |
| | Antibiotics | Dactinomycin | Choriocarcinoma, Wilms' tumor, |
| | | | rhabdomyosarcoma, testis, |
| | | | Kaposi's sarcoma |
| | | Daunorubicin | Acute granulocytic and acute |
| | | | lymphocytic leukemias |
| | | Doxorubicin | Soft-tissue, osteogenic, and other |
| | | 4′-Deoxydoxorubicin | sarcomas; Hodgkin's disease |
| | | | non-Hodgkin's lymphomas, acute |
| | | | leukemias, breast, |
| | | | genitourinary, thyroid, lung, |
| | | | stomach, neuroblastoma |
| | | | Bleomycin Testis, head |
| | | neck, skin, esophagus, | |
| | | lung, and genitourinary | |
| | | | tract; Hodgkin's disease, |
| | | | non-Hodgkin's lymphomas |
| | | Plicamycin | Testis, malignant hypercalcemia |
| | | Mitomycin | Stomach, cervix, colon, breast, |
| | | | pancreas, bladder, head and |
| | | | neck |
| | Enzymes | L-Asparaginase | Acute lymphocytic leukemia |
| | Taxanes | Docetaxel | Breast, ovarian |
| | Taxoids | Paclitaxel | |
| | Biological Response | Interferon Alfa | Hairy cell leukemia, Kaposi's |
| | Modifiers | | sarcoma, melanoma, carcinoid, |
| | | | renal cell, ovary, bladder, non- |
| | | | Hodgkin's lymphomas, mycosis |
| | | | fungoides, multiple myeloma, |
| | | | chronic granulocytic leukemia |
| | | | Tumor Necrosis |
| | | Factor | Investigational |
| | | Tumor-Infiltrating | Investigational Lymphocytes |
| Miscellaneous Agents | | Cisplatin | Testis, ovary, bladder, head and |
| Platinum Coordination | | | neck, lung, thyroid, cervix |
| | Complexes | Carboplatin | endometrium neuroblastoma, |
| | | | osteogenic sarcoma |
| | Anthracenedione | Mitoxantrone | Acute granulocytic leukemia, |
| | | | breast |
| | Substituted Urea | Hydroxyurea | Chronic granulocytic leukemia, |
| | | | polycythemia vera, essential |
| | | | thrombocytosis, malignant |
| | | | melanoma |
| | Methyl Hydrazine | Procarbazine | Hodgkin's disease Derivative |
| | Adrenocortical Suppressant | Mitotane | Adrenal cortex |
| | | Aminoglutethimide | Breast |
| Hormones and | Adrenocorti-costeroids | Prednisone | Acute and chronic lymphocytic |
| Antagonists | | | leukemias, non-Hodgkin's |
| | | | lymphomas, Hodgkin's disease, |
| | | | breast |
| | Progestins | Hydroxy-progesterone caproate | Endometrium, breast |
| | | Medroxy-progesterone acetate | Megestrol acetate |
| | Estrogens | Diethylstil-bestrol | Breast, prostate Ethinyl estradiol |
| | Antiestrogen | Tamoxifen | Breast |
| | Androgens | Testosterone | Breast |
| | | propionate Fluoxymesterone | |
| | Antiandrogen | Flutamide | Prostate |
| | Gonadotropin- | Leuprolide | Prostate, Estrogen-receptor- |
| | | Goserelin | positive breast releasing hormone |
| | | | analog |
| ′Adapted from Calabresi, P., and B. A. Chabner, “Chemotherapy of Neoplastic Diseases” Section XII, pp 1202-1263 in: Goodman and Gilman's The Pharmacological Basis Therapeutics, Eighth ed., 1990 Pergamin Press, Inc.; and Barrows, L. R., “Antineoplastic and Immunoactive Drugs”, Chapter 75, pp 1236-1262, in: Remington: The Science: Practice of Pharmacy, Mack Publishing Co. Easton, PA, 1995; both references are incorporated by reference herein, in particular treatment | | | |
| # protocols. | | | |

Modifications of the L-Canavanine molecule to afford structural analogs of L-arginine are based on the following considerations. First, since x-ray crystallographic studies have revealed that the interatomic distance between the beta-carbon and the carbon of the guanidino group of L-Canavanine is somewhat shorter than that in the L-arginine molecule, an insertion of an extra methylene group into the L-Canavanine molecule while retaining the important guanidinooxy functional group was considered to be an effective alteration which might result in an increase in affinity for the arginyl-tRNA synthetase active site. Similarly, the chain-shortened analog of L-Canavanine, in which only one methylene group is present in the molecule was evaluated.

Second, although it is reasonable to contend that the antitumor activity of L-Canavanine is stereospecific for the L-isomer, since arginyl-tRNA synthetase would undoubtedly recognize the L-enantiomer of arginine as a substrate, the biological activity of the D-enantiomer of canavanine has not been determined, and may also have desirble biological properties. D-2-Amino-3-(aminooxy)propionic acid dihydrochloride, D-2-Amino-3-(guanidinooxy)propionic acid, and D-2-Amino-4-[assym-N^{G}, N^{G}-dimethyl (guanidinooxy)] butanoic acid, and mixtures thereof, were pursued to determine if they exhibited MIA-PaCa-2 cell growth-inhibitory activity and to compare the activity of this D-stereoisomeric form with that of its naturally occurring L-antipode. It was believed that D-Canavanine would not be an arginyl-tRNA synthetase substrate; thus, any adverse effects noted with D-Canavanine could not result directly from its incorporation into newly synthesized protein. In this respect, the D-enantiomer offers a means of evaluating canavanine's activity divorced from its role in protein synthesis. Racemic forms also may have interesting properties that combine the effects of the L- and D-isomers.

Third, ionic and hydrogen-bonding interactions of the guanidino group of L-arginine with neighboring amino acid residues are crucial for establishing the three-dimensional structure of a protein; replacement of this moiety with the guanidinooxy moiety of L-Canavanine results in the formation of aberrant and dysfunctional protein. Thus, analogs in which the guanidinooxy group has been further modified appears to cause a greater deleterious effect on the tertiary/quatenary structure of L-arginyl-containing proteins than does L-Canavanine. Thus, the effect of structural alteration of the terminal guanidinooxy group of L-Canavanine was also evaluated.

Finally, the methyl, ethyl, isopropyl, n-propyl, n-butyl, and n-octyl esters of L-Canavanine exhibit greater lipophilicity than canavanine and appear to possess improved cell membrane penetration properties. These compounds can constitute prodrug candidate forms of L-Canavanine, because they can be metabolized in vivo, such as attacked by cytosolic esterases to generate the parent compound.

Hereinafter, a “prodrug” is a precursor (forerunner) of the sensitizer. A prodrug may undergo chemical conversion by metabolic processes to the parent drug, thus becoming an active sensitizer. For example, an ester of L-Canavanine, wherein the acidic proton of the carboxylic acid group of L-Canavanine may be replaced by CH₂CH₃, or CH₃, is such a prodrug. It may or may not be a sensitizer in its prodrug form. However, when it has been attacked by cytosolic esterases in vitro or in vivo, which convert the ester group to a carboxylic acid, the ester prodrug becomes a sensitizer, i.e., L-Canavanine.

The sensitizer may be represented by at least one of the following structures (a-n) of Formula 2 in its S—, R—, or racemic form:

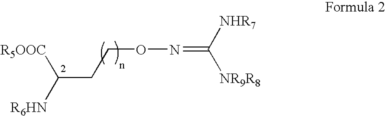

Examples 1-14 describe sensitizers represented by Formula 2.

### EXAMPLE 1

The sensitizer may be represented by Formula 2, wherein (a) R₅, R₆, R₇, R₈, and R₉═H; n=0; S or R configuration at carbon-2, or a racemic mixture.

### EXAMPLE 2

The sensitizer may be represented by Formula 2, wherein (b) R₅, R₆, R₇, R₈, and R₉═H; n=1; S or R configuration at carbon-2, or a racemic mixture.

### EXAMPLE 3

The sensitizer may be represented by Formula 2, wherein (c) R₅, R₆, R₇, R₈, and R₉═H; n=2; S or R configuration at carbon-2, or a racemic mixture.

### EXAMPLE 4

The sensitizer may be represented by Formula 2, wherein (d) R₅, R₆, R₇, R₈, and R₉═H; n=3; S or R configuration at carbon-2, or a racemic mixture.

### EXAMPLE 5

The sensitizer may be represented by Formula 2, wherein (e) R₅═CH₃; R₆, R₇, R₈, and R₉═H; n=0-3; S or R configuration at carbon-2, or a racemic mixture.

### EXAMPLE 6

The sensitizer may be represented by Formula 2, wherein (f) R₅═C₂H₅; R₆, R₇, R₈, and R₉═H; n=0-3; S or R configuration at carbon-2, or a racemic mixture.

### EXAMPLE 7

The sensitizer may be represented by Formula 2, wherein (g) R₅=n-C₃H₇; R₆, R₇, R₈, and R₉═H; n=0-3; S or R configuration at carbon-2, or a racemic mixture.

### EXAMPLE 8

The sensitizer may be represented by Formula 2, wherein (h) R₅=i-C₃H₇; R₆, R₇, R₈, and R₉═H; n=0-3; S or R configuration at carbon-2, or a racemic mixture.

### EXAMPLE 9

The sensitizer may be represented by Formula 2, wherein(i) R₅=n-C₄H₉; R₆, R₇, R₈, and R₉═H; n=0-3; S or R configuration at carbon-2, or a racemic mixture.

### EXAMPLE 10

The sensitizer may be represented by Formula 2, wherein (j) R₅=n-C₈H₁₇; R₆, R₇, R₈, and R₉═H; n=0-3; S or R configuration at carbon-2, or a racemic mixture.

### EXAMPLE 11

The sensitizer may be represented by Formula 2, wherein (k) R₅, R₆ and R₈ ═H; R₇—R₉═(CH₂—CH₂); n=0-3; S or R configuration at carbon-2, or a racemic mixture.

### EXAMPLE 12

The sensitizer may be represented by Formula 2, wherein (1) R₅, R₆, and R₉═H; R₇ and/or R₈═CH₃; n=0-3; S or R configuration at carbon-2, or a racemic mixture.

### EXAMPLE 13

The sensitizer may be represented by Formula 2, wherein (m) R₅, R₇, R₈, and R₉═H; R₆=benzoyl; n=0-3; S or R configuration at carbon-2, or a racemic mixture.

### EXAMPLE 14

The sensitizer may be represented by Formula 2, wherein (n) R₅═C₂H₅; R₆=benzoyl; R₇, R₈ and R₉═H; n=0-3; S or R configuration at carbon-2, or a racemic mixture.

Alternatively, the sensitizer may be a structural analog of L-arginine such as a compound having a Formula 3 in its S—, R—, or racemic form: 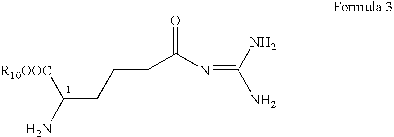
wherein R₁₀ comprises a hydrogen atom or a hydrocarbyl group with a primary, secondary, or a tertiary attachment points. The hydrocarbyl group may be an alkyl, an alkenyl, an alkynl, an aralkyl, an alkaryl or an aryl group.

The alkyl, alkenyl, alkynl, aralkyl, alkaryl or aryl groups may have from 1-20 carbon atoms.

The alkyl groups of the aralkyl or alkaryl groups may be linear, branched or cyclic and the aryl groups may have at least one C₃-C₈ carbon ring.

The sensitizer may be a structural analog of L-arginine, such as a compound having a Formula 4 in all enantiomeric, diastereomeric or racemic forms at carbon-2 and carbon-3, wherein each R₁₁, R₁₂ and R₁₃ independently at each occurrence may be a hydrogen atom, or a hydrocarbyl group with a primary, secondary or tertiary point of attachment, that includes an alkyl group, an alkenyl group, an alkynl group, an aralkyl group, an alkaryl group or an aryl group, wherein the alkyl, alkenyl, alkynyl, aralkyl, alkaryl or aryl groups may have from 1-20 carbon atoms, wherein the alkyl groups of the aralkyl, or alkaryl groups may be linear, branched or cyclic and the aryl groups may be at least one C₃-C₈ carbon ring. The sensitizer represented by Formula 4 is a prodrug ester when R₁₁ is not a hydrogen atom.

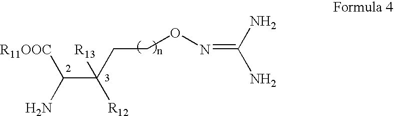

The sensitizer may be represented by at least one of the following structures (a-i) of Formula 4: (a) R₁₁ and R₁₂═H; R₁₃═NH₂; n=0-3, (b) R₁₁ and R₁₂═H; R₁₃═NH₂; n=0-3, (c) R₁₁ and 13═H; R₁₂═NH₂; n=0-3, (d) R₁₁=methyl; R₁₂═H; and R₁₃═NH₂; n=0-3, (e) R₁₁=ethyl; R₁₂═H; and R₁₃═NH₂; n=0-3, (f) R₁₁=isopropyl; R₁₂═H; and R₁₃═NH₂; n=0-3, (g) R₁₁=n-propyl; R₁₂═H; and R₁₃═NH₂; n=0-3, (h) R₁₁=n-butyl R₁₂═H; and R₁₃═NH₂; n=0-3, (i) R₁=n-octyl; R₁₂═H; and R₁₃═NH₂; n=0-3

The sensitizer may be a structural analog of L-arginine, such as a compound having a Formula 4 in all enantiomeric, diastereomeric or racemic forms at carbon-2 and carbon-3, wherein each R₁₄, R₁₅ and R₁₆ independently at each occurrence may be a hydrogen atom, or a hydrocarbyl group with a primary, secondary or tertiary point of attachment, that includes an alkyl, an alkenyl, an alkynl, an aralkyl, an alkaryl or an aryl group, wherein the alkyl, alkenyl, alkynl, aralkyl, alkaryl or aryl groups may have from 1-20 carbon atoms, wherein the alkyl groups of the aralkyl, or alkaryl groups may be linear, branched or cyclic and the aryl groups may be at least one C₃-C₈ carbon ring. The sensitizer represented by Formula 5 is a prodrug ester when R₁₁ is not a hydrogen atom.

The sensitizer may be represented by at least one of the following structures (a-i) of Formula 5: (a) R₁₄ and R₁₅═H; R₁₆═NH₂; n=0-3; 2R, 3S; (b) R₁₄ and R₁₅═H; R₁₆═NH₂; n=0-3; 2R, 3S; (c) R₁₄ and R₁₆═H; R₁₅═NH₂; n=0-3; 2R, 3R; (d) R₁₄ and R₁₅═H; R₁₆═NH₂; n=0-3; 2R, 3S ; (e) R₁₄=ethyl; R₁₅═H; and R₁₆═NH₂; n=0;2R, 3S; (f) R₁₄=isopropyl; R₁₅═H; and R₁₆═NH₂; n=0-3; 2R, 3S; (g) R₁₄=n-propyl; R₁₅═H; and R₁₆═NH₂; n=0-3; 2R, 3S; (h) R₁₄=n-butyl; R₁₅═H; and R₁₆═NH₂; n=0-3; 2R, 3S; and (i) R₁₄=n-octyl; R₁₅═H; and R₁₆═NH₂; n=0-3; 2R, 3S.

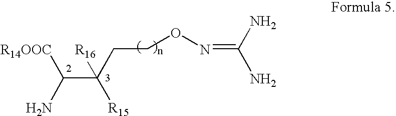

The sensitizer may represented by at least one of structures (a-h) of Formula 6: (a) R₁₇ and R₁₉═H; R₁₈═NH₂; n=0-3; 2R, 3R, (b) R₁₇ and R₁₉═H; R₁₈═NH₂; n=0-3; 2R, 3R, (c) R₁₇=methyl; R₁₉═H; and R₁₈═NH₂; n=0-3; 2R, 3R, (d) R₁₇=ethyl; R₁₉═H; and R₁₈═NH₂; n=0-3; 2R, 3R, (e) R₁₇=isopropyl; R₁₉═H; and R₁₈═NH₂; n=0-3; 2R, 3R, (f) R₁₇=n-propyl; R₁₉═H; and R₁₈═NH₂; n=0-3; 2R, 3R, (g) R₁₇=n-butyl; R₁₉═H; and R₁₈═NH₂; n=0-3; 2R, 3R and (h) R₁₇=n-octyl; R₁₉═H; and R₁₈═NH₂; n=0-3; 2R, 3R.

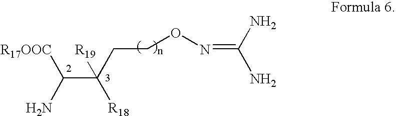

The sensitizer may be represented by at least one of structures (a-h) of Formula 7: (a) R₂₀ and R₂₂═H; R₂₁═NH₂; n=0-3; 2S, 3R, (b) R₂₀ and R₂₂═H; R₂₁═NH₂; n=0-3; 2S, 3R, (c) R₂₀=methyl; R₂₂═H; and R₂₁═NH₂; n=0-3; 2S, 3R, (d) R₂₀=ethyl; R₂₂═H; and R₂₁═NH₂; n=0-3; 2S, 3R, (e) R₂₀=isopropyl; R₂₂═H; and R₂₁═NH₂; n=0-3; 2S, 3R, (f) R₂₀=n-propyl; R₂₂═H; and R₂₁═NH₂; n=0-3; 2S, 3R, (g) R₂₀=n-butyl; R₂₂═H; and R₂₁═NH₂; n=0-3; 2S, 3R, and (h) R₂₀=n-octyl; R₂₂═H; and R₂₁═NH₂; n=0-3; 2S, 3R.

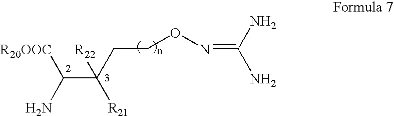

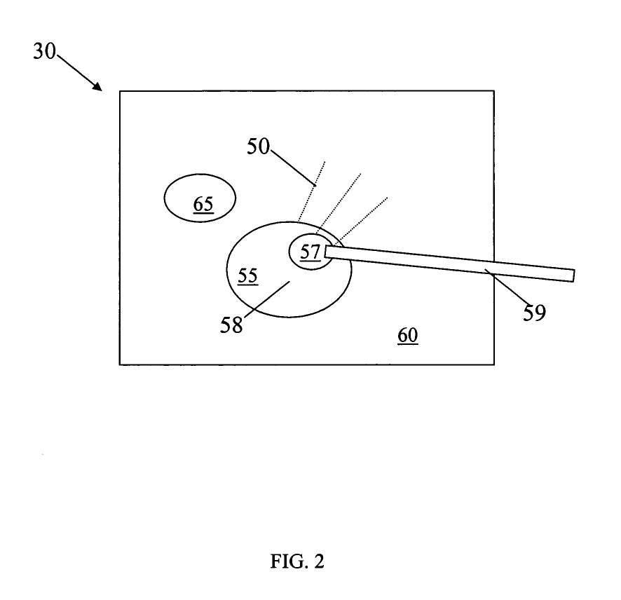

FIG. 2 depicts an apparatus 30, comprising a body 60, for example, a person's body, having cancerous tissue 57, such as a tumor, in an organ 55. The apparatus 30 may further comprise a sensitizer delivering device 59, such as for example, a sensitizer delivering device, for in vivo or in vitro delivering a sensitizer to a patient that has cancer, as in the step 10 of the method 1 as depicted in FIG. 1, supra. The apparatus 30 may include a radiation source 50 for exposing the cancerous tissue 57, such as the tumor, in the organ 55 in the body 60, as in step 20 of the method 1. The organ 55 may be a human pancreas, wherein the organ 55 may be inflicted with pancreatic cancer. Delivering the sensitizer may be achieved by injecting the sensitizer using the sensitizer delivering device 59, as depicted in FIG. 2, in accordance with the step 10 of the method 1. The sensitizer delivering device 59 may be dedicated to administering the sensitizer or it may also be used to administer radioactive materials to expose cancerous tissue 57, as in step 20 of the method 1.

Methods of delivery, as in the step 10 of the method 1 comprise systemic administration to humans and animals in unit dosage forms, such as oral or sublingual tablets, capsules, pills, powders, granules, suppositories, pessaries, sterile parenteral solutions or suspensions, sterile non-parenteral solutions or suspensions oral solutions or suspensions, oil in water or water in oil emulsions, parenteral solutions or suspensions, incorporation into slow release matrices, transdermal delivery devices, wherein the dosage contains suitable quantities of an active ingredient. A dosage for mammals may be from about 25 to 50 mg per kilogram body weight is administered per day. When the dosage is administered parenterally, such as intramuscularly, the dosage for mammals maybe about 0.1-30 mg per kilogram of body weight per day. Hereinafter, parenterally means located outside the alimentary canal. A dosage for mammals may be from about 0.1 to about 25 mg per kilogram body weight is administered per day. Parenterally may also mean taken into the body or administered in a manner other than through the digestive tract, as by intravenous or intramuscular injection.

Referring to FIG. 1, the step 20 of the method 1, exposing the patient to radiation may be achieved using any of the following appropriate methods. Effective radiotherapy needs to maximize exposure of the affected tissues 57 while sparing normal surrounding tissues 58. In one embodiment, the exposing the patient to radiation step 20 may be interstitial therapy, where needles 59 containing a radioactive source are embedded in the tumor 57, has become a valuable new approach. In this way, large doses of irradiation can be delivered locally while sparing the surrounding normal structures, 58 and 65. Alternatively, the exposing the patient to radiation step 20 may be intraoperative radiotherapy, where the beam 50 is placed directly onto the tumor 57 in the organ 55 during surgery while normal structures 65 are moved safely away from the beam 50. Again, this achieves effective irradiation of the tumor 57 while limiting exposure to surrounding normal structures, 58 and 65.

Radiotherapy, also called radiation therapy, is the treatment of cancer and other diseases with ionizing radiation. Ionizing radiation deposits energy that injures or destroys cells in the area being treated (the “target tissue”) by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or uterine cervix. It can also be used to treat leukemia and lymphoma (cancers of the blood-forming cells and lymphatic system, respectively).

One type of radiation therapy commonly used involves photons, “packets” of energy. X-rays were the first form of photon radiation to be used to treat cancer. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy.

Gamma rays are another form of photons used in radiotherapy. Gamma rays are produced spontaneously as certain elements (such as radium, uranium, and cobalt 60) release radiation as they decompose, or decay. Each element decays at a specific rate and gives off energy in the form of gamma rays and other particles. X-rays and gamma rays have the same effect on cancer cells.

Another technique for delivering radiation to cancer cells is to place radioactive implants directly in a tumor or body cavity. This is called internal radiotherapy. (Brachytherapy, interstitial irradiation, and intracavitary irradiation are types of internal radiotherapy.) In this treatment, the radiation dose is concentrated in a small area, and the patient stays in the hospital for a few days. Internal radiotherapy is frequently used for cancers of the tongue, uterus, and cervix.

Several new approaches to radiation therapy are being evaluated to determine their effectiveness in treating cancer. One such technique is intraoperative irradiation, in which a large dose of external radiation is directed at the tumor and surrounding tissue during surgery.

Another investigational approach is particle beam radiation therapy. This type of therapy differs from photon radiotherapy in that it involves the use of fast-moving subatomic particles to treat localized cancers. A very sophisticated machine is needed to produce and accelerate the particles required for this procedure. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation.

Scientists also are looking for ways to increase the effectiveness of radiation therapy. Two types of investigational drugs are being studied for their effect on cells undergoing radiation. Sensitizers make the tumor cells more likely to be damaged, and radioprotectors protect normal tissues from the effects of radiation. Hyperthermia, the use of heat, is also being studied for its effectiveness in sensitizing tissue to radiation.

Other recent radiotherapy research has focused on the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells. The success of this technique will depend upon both the identification of appropriate radioactive substances and determination of the safe and effective dose of radiation that can be delivered in this way.

Radiation therapy may be used alone or in combination with chemotherapy or surgery. Like all forms of cancer treatment, radiation therapy can have side effects. Possible side effects of treatment with radiation include temporary or permanent loss of hair in the area being treated, skin irritation, temporary change in skin color in the treated area, and tiredness. Other side effects are largely dependent on the area of the body that is treated.

The sensitizer may be a component of a pharmaceutical composition comprising a sensitizer and a pharmaceutically acceptable carrier. The sensitizer in the pharmaceutical composition may be selected from the group consisting of D-2-Amino-3-(aminooxy)propionic acid dihydrochloride; D-2-Amino-3-(guanidinooxy)propionic acid; L-2-Amino-4-[assym-N^{G}, N^{G}-dimethyl (guanidinooxy)] butanoic acid, and mixtures thereof. The sensitizer in the pharmaceutical composition may be derived from a prodrug that may be selected from the group consisting of L-Canavanine esters, methyl L-2-amino-4-guanidinooxybutanoate, ethyl L-2-amino-4-guanidinooxybutanoate, Isopropyl L-2-amino-4-guanidinooxybutanoate, n-propyl L-2-amino-4-guanidinooxybutanoate, n-butyl L-2-amino-4-guanidinooxybutanoate, n-octyl-4-guanidinooxybutanoate, and mixtures thereof. The pharmaceutical composition may advantageously include 5-fluorouracil. The pharmaceutical composition may advantageously include a compound that may be selected from the group consisting of (S)-2-aminoethyl-L-cysteine, L-2-azetidine carboxylic acid, L-selenomethionine, L-3-[N-hydroxy-4-oxypyridyl]-2-amino-propionic acid and mixtures thereof.

In addition to the sensitizer, the pharmaceutical composition of the present invention may also include various other pharmaceutically acceptable components as additives or adjuncts. Pharmaceutically acceptable components as additives or adjuncts which may be employed in relevant circumstances include antioxidants, free radical scavenging agents, peptides, growth factors, antibiotics, bacteriostatic agents, immunosuppressives, anticoagulants, buffering agents, anti-inflammatory agents, anti-pyretics, time release binders, anaesthetics, steroids and corticosteroids. Such components can provide additional therapeutic benefit, such as to affect the therapeutic action of the prodrugs, or act towards preventing any potential side effects that may be posed as a result of administration of the prodrugs. In certain circumstances, a compound of the present invention can be employed as part of a prodrug with other compounds intended to prevent or treat cancer.

Acceptable carriers for the purpose of this invention are carriers that do not adversely affect the sensitizer, the host, or the material comprising the sensitizer delivery device. Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving, stabilizing, wetting, emulsifying agents and the like together with the sensitizer of this invention.

The sensitizers can advantageously be used as adjunct therapy in combination with existing therapies, such as hyperthermia, in the management cancer treatment in patients having cancer.

The foregoing description of the embodiments of this invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of this invention as defined by the accompanying.

We claim:

1. A method, comprising:
delivering in vivo a sensitizer to a patient that has cancer; and
exposing the patient to a chemotherapeutic agent.

2. The method of claim 1, wherein the sensitizer is represented by Formula 1 in its S—, R—, or racemic form, as follows: 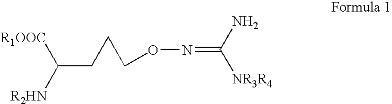
wherein each R₁, R₃ and R₄ independently at each occurrence comprises a hydrogen atom or a hydrocarbyl group with a primary, a secondary or a tertiary carbon attachment point, said hydrocarbyl group selected from the group consisting of an alkyl group, an alkenyl group, an alkynl group, an aralkyl group, an alkaryl and an aryl group,
wherein each alkyl, alkenyl, alkynl, aralkyl, alkaryl or aryl groups has from 1-20 carbon atoms,
wherein the alkyl groups of the aralkyl, or alkaryl groups may be linear, branched or cyclic and the aryl groups may be at least one C₃ -C₈ carbon ring, and
wherein each R₂ and R₃ independently at each occurrence comprises a hydrogen atom or a carbonyl, said carbonyl selected from the group consisting of tert-butyloxycarbonyl (BOC-) and benzoyl (Bz-).

3. The method of claim 1, further comprising delivering the sensitizer as L-Canavanine esters.

4. The method of claim 1, further comprising delivering the sensitizer as a dihydrohalide salt and/or as admixtures of the acid with a salt-forming material.

5. The method of claim 4, wherein said L-Canavanine Esters are selected from the group consisting of methyl, ethyl, isopropyl and n-propyl esters of L-Canavanine and combinations thereof.

6. The method of claim 1, wherein said cancer is pancreatic cancer.

7. The method of claim 1, wherein a dose of the sensitizer is from about 0.1 to about 25 mg per kilogram body weight a day.

8. The method of claim 1, wherein the sensitizer is represented by at least one of the following structures (a-n) of Formula 2 in its S—, R—, or racemic form: 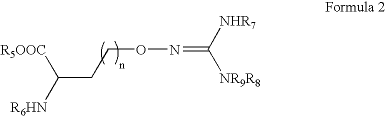
wherein
(a) R₅, R₆, R₇, R₈, and R₉═H; n=0; S or R configurations at carbon 2, or a racemic mixture of R and S enantiomers,
(b) R₅, R₆, R₇, R₈, and R₉═H; n=1; S or R configurations at carbon 2, or a racemic mixture of R and S enantiomers,
(c) R₅, R₆, R₇, R₈, and R₉═H; n=2; S or R configurations at carbon 2, or a racemic mixture of R and S enantiomers,
(d) R₅, R₆, R₇, R₈, and R₉═H; n=3; S or R configurations at carbon 2, or a racemic mixture of R and S enantiomers,
(e) R₅═CH₃; R₆, R₇, R₈, and R₉═H; n=0-3; S or R configurations at carbon 2, or a racemic mixture of R and S enantiomers,
(f) R₅═C₂H₅; R₆, R₇, R₈, and R₉═H; n=0-3; S or R configurations at carbon 2, or a racemic mixture of R and S enantiomers,
(g) R₅=n-C₃H₇; R₆, R₇, R₈, and R₉═H; n=0-3; S or R configurations at carbon 2, or a racemic mixture of R and S enantiomers,
(h) R₅=i-C₃H₇; R₆, R₇, R₈, and R₉═H; n=0-3; S or R configurations at carbon 2, or a racemic mixture of R and S enantiomers,
(i) R₅=n-C₄H₉; R₆, R₇, R₈, and R₉═H; n=0-3; S or R configurations at carbon 2, or a racemic mixture of R and S enantiomers,
(j) R₅=n-C₈H₁₇; R₆, R₇, R₈, and R₉═H; n=0-3; S or R configurations at carbon 2, or a racemic mixture of R and S enantiomers,
(k) R₅, R₆, and R₈═H; (R₇—R₉)═(CH₂—CH₂); n=0-3; S or R configurations at carbon 2, or a racemic mixture of R and S enantiomers,
(l) R₅, R₆, and R₉═H; R₇ and/or R₈═—CH₃; n=0-3; S or R configurations at carbon 2, or a racemic mixture of R and S enantiomers,
(m) R₅, R₇, R₈, and R₉═H; R₆=-Bz (—C₆H₅CO=-Bz); n=0-3; S or R configurations at carbon 2, or a racemic mixture of R and S enantiomers, and
(n) R₅═C₂H₅; R₆=-Bz (—C₆H₅CO=-Bz); R₇, R₈ and R₉═H; n=0-3; S or R configurations at carbon 2, or a racemic mixture of R and S enantiomers.

9. The method of claim 9, wherein a dose of the sensitizer is from about 25 mg to about 50 mg per kilogram body weight a day.

10. The method of claim 1, wherein the sensitizer is represented by Formula 3 in its S—, R—, or racemic form, as follows: 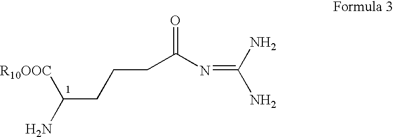
wherein R₁₀ comprises a hydrogen atom or a hydrocarbyl group with a primary, secondary, or a tertiary attachment point, said hydrocarby group selected from the group consisting of an alkyl group, an alkenyl group, an alkynl group, an aralkyl group, an alkaryl group or an aryl group,
wherein the alkyl, alkenyl, alkynl, aralkyl, alkaryl or aryl groups have from 1-20 carbon atoms,
wherein the alkyl groups of the aralkyl, or alkaryl groups are linear, branched or cyclic and the aryl groups have at least one C3 -C8 carbon ring.

11. The method of claim 11, wherein a dose of the sensitizer is from about 0.1 to about 25 mg per kilogram body weight a day.

12. The method of claim 1, wherein the sensitizer is delivered in combination with hyperthermia therapy.

13. The method of claim 1, wherein the sensitizer is delivered in combination with adjuncts.

14. The method of claim 14, wherein the adjuncts are selected from the group consisting of antioxidants, free radical scavenging agents, peptides, growth factors, antibiotics, bacteriostatic agents, immunosuppressives, anticoagulants, buffering agents, anti-inflammatory agents, anti-pyretics, time release binders, anaesthetics, steroids, corticosteroids, and combinations thereof.

15. The method of claim 1, further comprising delivering systemic administration to humans or animals in unit dosage forms selected from the group consisting of tablets, capsules, pills, powders, granules, suppositories, sterile parenteral solutions or suspensions, sterile non-parenteral solutions or suspensions, oral solutions or suspensions, oil in water emulsions or water in oil emulsions, and combinations thereof.

16. The method of claim 16, wherein parenteral solutions or suspensions are incorporated in a slow release matrix for administering transdermally.

17. The method of claim 9, wherein a dosage for mammals is about 0.1 to 25 mg per kilogram body weight is administered per day.

18. The method of claim 9, wherein a dosage of about 0.1 to about 30 mg per kilogram of body weight per day is administered intramuscularly.

19. The method of claim 1, wherein the chemotherapeutic agent is radiation.

20. The method of claim 1, wherein the chemotherapeutic agent includes alkylating agents.

21. A pharmaceutical composition comprising a sensitizer and a pharmaceutically acceptable carrier.

22. The pharmaceutical composition according to claim 21, wherein the sensitizer is selected from the group consisting of D-2-Amino-3-(aminooxy)propionic acid dihydrochloride; D-2-Amino-3-(guanidinooxy)propionic acid; L-2-Amino-4-[assym-N^{G}, N^{G}-dimethyl (guanidinooxy)] butanoic acid, and mixtures thereof.

23. The pharmaceutical composition according to claim 21, wherein the sensitizer is derived from a prodrug selected from the group consisting of L-Canavanine esters, methyl L-2-amino-4-guanidinooxybutanoate, ethyl L-2-amino-4-guanidinooxybutanoate, isopropyl L-2-amino-4-guanidinooxybutanoate, n-propyl L-2-amino-4-guanidinooxybutanoate, n-butyl L-2-amino-4-guanidinooxybutanoate, n-octyl-4-guanidinooxybutanoate, and mixtures thereof.

24. The pharmaceutical composition according to claim 21, wherein said composition further comprises 5-fluorouracil.

25. The pharmaceutical composition according to claim 21, wherein said composition further comprises a compound selected from the group consisting of (S)-2-aminoethyl-L-cysteine, L-2-azetidine carboxylic acid, L-selenomethionine, L-3-[N-hydroxy-4-oxypyridyl]-2-amino-propionic acid and mixtures thereof.

26. The composition of claim 21, wherein the pharmaceutically acceptable carriers are selected from the group consisting of sterile water; saline, dextrose, dextrose in water or saline, condensation products of castor oil and ethylene oxide combining about 30 to about 35 moles of ethylene oxide per mole of castor oil, liquid acid, lower alkanols, corn oil, peanut oil, and sesame oil.

27. The composition of claim 21, wherein the pharmaceutically acceptable carriers comprise emulsifiers, said emulsifiers selected from the group consisting of mono- or di-glyceride of a fatty acid, a phosphatide, wherein the phosphatide includes lecithin, and glycols, wherein the glycols include polyalkylene glycols, wherein the pharmaceutically acceptable carriers are in aqueous media in the presence of a suspending agent, wherein the suspending agent is selected from the group consisting of sodium carboxymethylcellulose, sodium alginate, and poly(vinylpyrolidone) and/or suitable dispensing agents, wherein the dispensing agents are selected from the group consisting of lecithin and polyoxyethylene stearate.

28. The composition of 21, wherein the pharmaceutically acceptable carriers contain adjuvants, wherein the adjuvents are selected from the group consisting of preserving agents, stabilizing agents, wetting agents, emulsifying agents and combinations thereof.

29. A method, comprising:
delivering in vivo a dose of a sensitizer to a patient that has cancer, wherein the sensitizer is represented by at least one of structures (a-i) of Formula 4 in its S—, R—, or racemic form: 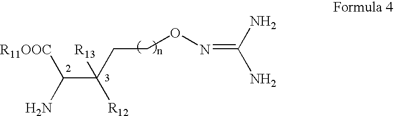
wherein
(a) R₁₁ and R₁₂═H; R₁₃═NH₂; n=0-3
(b) R₁₁ and R₁₂═H; R₁₃═NH₂; n=0-3
(c) R₁₁ and R₁₃═H; R₁₂═NH₂; n=0-3
(d) R₁₁=methyl; R₁₂═H; and R₁₃═NH₂; n=0-3
(e) R₁₁=ethyl; R₁₂═H; and R₁₃═NH₂; n=0-3
(f) R₁₁=isopropyl; R₁₂═H; and R₁₃═NH₂; n=0-3
(g) R₁₁=n-propyl; R₁₂═H; and R₁₃═NH₂; n=0-3
(h) R₁₁=n-butyl; R₁₂═H; and R₁₃═NH₂; n=0-3
(i) R₁₁=n-octyl; R₁₂═H; and R₁₃═NH₂; n=0-3 and
exposing the patient to a chemotherapeutic agent.

30. The method of claim 29, wherein a dose of the sensitizer is from about 0.1 to about 25 mg per kilogram body weight a day.

31. The method of claim 29, wherein the chemotherapeutic agent is radiation.

32. A method, comprising:
delivering in vivo a dose of a sensitizer to a patient that has cancer, wherein the sensitizer is represented by structures (a-i) of Formula 5: 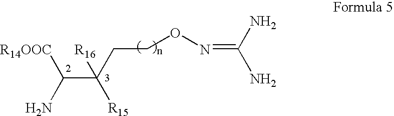
wherein
(a) R₁₄ and R₁₅═H; R₁₆═NH₂; n=0-3; 2R, 3S
(b) R₁₄ and R₁₅═H; R₁₆═NH₂; n=0-3; 2R, 3S
(c) R₁₄ and R₁₆═H; R₁₅═NH₂; n=0-3; 2R, 3R
(d) R₁₄ and R₁₅═H; R₁₆═NH₂; n=0-3; 2R, 3S
(e) R₁₄=ethyl; R₁₅═H; and R₁₆═NH₂; n=0; 2R, 3S
(f) R₁₄=isopropyl; R₁₅═H; and R₁₆═NH₂; n=0-3; 2R, 3S
(g) R₁₄=n-propyl; R₅═H; and R₁₆═NH₂; n=0-3; 2R, 3S
(h) R₁₄=n-butyl; R₁₅═H; and R₁₆═NH₂; n=0-3; 2R, 3S
(i) R₁₄=n-octyl; R₁₅═H; and R₁₆═NH₂; n=0-3; 2R, 3S and
exposing the patient to a chemotherapeutic agent.

33. The method of claim 32, wherein the chemotherapeutic agent is radiation.

34. A method, comprising:
delivering in vivo a dose of a sensitizer to a patient that has cancer, wherein the sensitizer is represented by at least one of structures (a-h) of Formula 6: 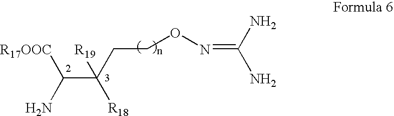
wherein
(a) R₁₇ and R₁₉═H; R₁₈═NH₂; n=0-3; 2R, 3R
(b) R₁₇ and R₁₉═H; R₁₈═NH₂; n=0-3; 2R, 3R
(c) R₁₇=methyl; R₁₉═H; and R₁₈═NH₂; n=0-3; 2R, 3R
(d) R₁₇=ethyl; R₁₉═H; and R₁₈═NH₂; n=0-3; 2R, 3R
(e) R₁₇=isopropyl; R₁₉═H; and R₁₈═NH₂; n=0-3; 2R, 3R
(f) R₁₇=n-propyl; R₁₉═H; and R₁₈═NH₂; n=0-3; 2R, 3R
(g) R₁₇=n-butyl; R₁₉═H; and R₁₈═NH₂; n=0-3; 2R, 3R
(h) R₁₇=n-octyl; R₁₉═H; and R₁₈═NH₂; n=0-3; 2R, 3R; and
exposing the patient to a chemotherapeutic agent.

35. A method, comprising:
delivering in vivo a dose of a sensitizer to a patient that has cancer, wherein the sensitizer is represented by at least one of structures (a-h) of Formula 7: 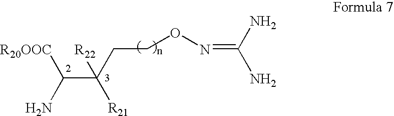
wherein
(a) R₂₀ and R₂₂═H; R₂₁═NH₂; n=0-3; 2S, 3R
(b) R₂₀ and R₂₂═H; R₂₁═NH₂; n=0-3; 2S, 3R
(c) R₂₀=methyl; R₂₂═H; and R₂₁═NH₂; n=0-3; 2S, 3R
(d) R₂₀=ethyl; R₂₂═H; and R₂₁═NH₂; n=0-3; 2S, 3R
(e) R20₁=isopropyl; R₂₂═H; and R₂₁═NH₂; n=0-3; 2S, 3R
(f) R₂₀=n-propyl; R₂₂═H; and R₂₁═NH₂; n=0-3; 2S, 3R
(g) R₂₀=n-butyl; R₂₂═H; and R₂₁═NH₂; n=0-3; 2S, 3R
(h) R₂₀ n-octyl; R₂₂═H; and R₂₁═NH₂; n=0-3; 2S, 3R; and
exposing the patient to a chemotherapeutic agent.
